(19) United States
(12) Patent Application Publication
(10) Pub. No.: US 2015/0147419 A1
(43) Pub. Date: May 28, 2015

(54) Compositions of Melicope Elleryana

(71) Applicant: Avon Products, Inc., Suffern, NY (US)

(72) Inventors: John W. Lyga, Basking Ridge, NJ (US); Thomas J. Maniscalco, Danbury, CT (US)

(21) Appl. No.: 14/335,467

(22) Filed: Jul. 18, 2014

Related U.S. Application Data
(60) Provisional application No. 61/908,545, filed on Nov. 25, 2013.

(51) Int. Cl. A61K 8/97

(52) U.S. Cl. CPC A61K 8/97
USPC 424/769

(57) ABSTRACT

Compositions comprising extracts from the Melicope elleryana plant are disclosed. In some embodiments, the extracts low odor extracts suitable for use in topical cosmetic formulations.

### CROSS-REFERENCE TO RELATED APPLICATIONS

This patent application claims priority to U.S. Patent Application Ser. No. 61/908,545, filed on Nov. 25, 2013. The entirety of the aforementioned application is incorporated herein in its entirety by reference.

### FIELD OF INVENTION

The present invention relates generally to topical formulations comprising extracts obtained from the plant Melicope elleryana. In one embodiment, the invention relates to the use of extracts of Melicope elleryana characterized by a diminished concentration of certain constituents that may contribute to off odor. The invention also relates to topical formulations comprising novel extracts of Melicope elleryana and topical formulations comprising extracts of Melicope elleryana prepared by new extraction methods. The extracts are useful as components of cosmetic and therapeutic compositions for topical application to human integuments.

### BACKGROUND

There has been significant interest in the use of botanical extracts in cosmetic and personal care compositions. Typically, botanical extracts are prepared from whole plants or portions thereof (e.g., leaves, flowers, bark, roots, etc.). The plant material is extracted with a suitable solvent, depending on the desired constituents to be isolated. One technique for preparing botanical extracts is steam distillation, in which water or steam is introduced into the plant material in the distillation apparatus and heated to increase the total vapor pressure of the system and thereby reduce the boiling point of the organic constituents. Steam distillation thus permits isolation of organic constituents at reduced temperature to avoid possible decomposition. In other techniques, the plant material is contacted with an organic solvent, such as ethanol, methanol, ethyl acetate, hexane, etc., optionally at elevated temperature, to isolate organic constituents from the plant. Extraction of plants with supercritical CO₂ has also been described.

These extraction techniques typically result in a large number of distinct organic constituents being removed from the plant due to their solubility in the solvent system or their similar boiling points. In some cases, these techniques may produce plant extracts that have undesirably properties, such as an objectionable color, odor, or toxicological profile.

Melicope elleryana is a species of flowering tree in the Melicope genus that may be found in the wild in rainforest areas of Australia (from the Clarence River in New South Wales to tropical northern Australia), New Guinea, and the Solomon Islands. The tree has opposite leaflets are in threes, mostly ovate. 6 to 13 cm long, tapering to a blunt point at the tip. Melicope elleryana is also known as Pink Flowered Doughwood. It has not previously been reported that extracts of Melicope elleryana may have objectionable odor profiles, however, the present invention is premised on the discovery that extracts of Melicope elleryana obtained by steam distillation may have strong, off odors that render the extracts less suitable for cosmetic purposes.

It is therefore an object of the present invention to provide extracts of Melicope elleryana that offer pleasant or neutral odor profiles, or that have less objectionable odor profiles than extracts of Melicope elleryana prepared by prior art steam distillations. It is another object of the invention to identify one or more constituents of extracts of Melicope elleryana that may contribute to objectionable odor profiles. It is a further object of the invention to provide extracts of Melicope elleryana that have reduced levels of one or more constituents that may contribute to objectionable odor profiles. It is yet another object of the invention to provide topical compositions (cosmetic or therapeutic) comprising extracts of Melicope elleryana that have acceptable odor profiles. It is yet another object of the invention to provide topical compositions (cosmetic or therapeutic) comprising extracts of Melicope elleryana that are prepared by new extraction methods.

The foregoing discussion is presented solely to provide a better understanding of nature of the problems confronting the art and should not be construed in any way as an admission as to prior art.

### SUMMARY OF THE INVENTION

In accordance with the foregoing objectives and others, the present invention provides extracts of Melicope elleryana that have pleasant or neutral odor profiles, or that have diminished “off odor” as compared to prior art extracts of Melicope elleryana, particularly those prepared by prior art steam distillation processes. The present invention is premised on the observation that certain extracts Melicope elleryana, for example, those prepared by steam distillation wherein plant material is combined with water in a heated vessel and all vapors are condensed and collected, may be characterized by an objectionable odor profile (“off odor”) than is unacceptable or undesirable to consumers of cosmetic and personal care products. Through extensive investigation, the inventors have identified certain organic compounds present in extracts of Melicope elleryana that may contribute to off odor and/or may separate with the off-odor causing fractions when an organic extract is distilled, redistilled, fractionated or otherwise processed, including, without limitation, 2-methyl-3-buten-2-ol, 3-methyl butanal, and 2-methyl-2-butenal. However, it will be understood that the invention is not limited to the removal or reduction of those compounds, but rather contemplates the improvement in odor by the removal of other individual or complex mixtures of volatile components as well. Moreover, extracts of Melicope elleryana prepared by the inventive methods described herein are intended to be within the scope of the invention regardless of whether a change in the odor profile is achieved.

In one aspect of the invention, extracts of Melicope elleryana (e.g., those containing zierone and/or evodone as the two most abundant constituents) are provided which have had some fraction of volatile constituents removed or reduced in amount. The extracts may be processed to remove volatile constituents, by for example, distillation, evaporation, chromatography, adsorption, and/or chemisorption.

In one aspect of the invention, extracts of Melicope elleryana (e.g., those containing zierone and/or evodone as the two most abundant constituents) are provided which are characterized by a reduced (e.g., reduced by at least about 25%, 50%, 75% or more by weight) or substantially eliminated (e.g., not detectable by GC of headspace) content of one of more volatile compounds (e.g., any of 2-methyl-3-buten-2-ol, 3-methyl butanal, or 2-methyl-2-butenal), as compared to an extract of Melicope elleryana obtained by steam distillation in which the entire distillate (e.g., including the front end) is collected and combined.

In another aspect of the invention, a method for preparing an extract of Melicope elleryana is provided comprising heating the whole plant or a portion thereof (e.g., leaves, terminal branchlets, etc.) in boiling water and/or in the presence of steam, condensing vapors generated thereby, and collecting at least portion of the organic distallate (e.g., a portion of the distillate containing zierone and/or evodone as the most abundant constituents), wherein a fraction of vapor or distillate comprising an amount of a volatile compound (e.g., a compound selected from 2-methyl-3-buten-2-ol, 3-methyl butanal, and/or 2-methyl-2-butenal) which is greater the than the amount of the same compound in any other fraction is discarded or not condensed. Typically, the fraction of vapor or distillate that is richest in the volatile compound is discarded or not condensed.

In further aspect of the invention, a method for preparing an extract of Melicope elleryana is provided comprising providing a first extract of Melicope elleryana (e.g., one containing zierone and/or evodone as the most abundant constituents), and preparing a second extract therefrom having a reduced amount of a volatile compound (e.g., a compound selected from 2-methyl-3-buten-2-ol, 3-methyl butanal, and/or 2-methyl-2-butenal) as compared to the amount of the volatile compound in the first extract. The second extract may be prepared, for example, by heating the first extract to a temperature sufficient to preferentially volatilize at least one of the selected compounds in the extract. The second extract may also be prepared, for example, by subjecting the first extract to reduced pressures (e.g., vacuum or partial vacuum) to preferentially volatilize at least one of the selected compound in the extract. The second extract also may be prepared, for example, by subjecting the first extract to a separation step (e.g., chromatography, chemisorption, distillation, etc.) to remove or reduce the amount of at least one of the selected compound in the extract.

In yet another aspect of the invention, topical formulations are provided comprising extracts of Melicope elleryana (e.g., ones containing zierone and/or evodone as the most abundant constituents) having a reduced amounts of one or more volatile compounds (e.g., compounds selected from 2-methyl-3-buten-2-ol, 3-methyl butanal, and/or 2-methyl-2-butenal). The topical formulations may be capable of improving one or more signs of dermatological aging when topically applied to human integuments (skin, lips, nails, hair, etc.), particularly skin. The topical formulations of the invention may, without limitation, reduce inflammation in the skin and/or inhibit calcineurin in the skin and/or lead to increased pro-collagen and/or collagen production within dermal fibroblasts and/or reduce lipid accumulation and/or increase lipolysis in adipocytes and/or reduce sebum production. The topical formulations will typically comprise one or more of a cosmetically acceptable vehicle, a film forming polymer, a thickener or rheology modifier, a pH adjuster, a preservative, an emulsifier, a gelling agent, an antioxidant, a humectant, an emollient, a fragrance, a colorant, and the like. The vehicle may comprise an emulsion, for example, a water-in-oil, oil-in-water, silicone-in-water, or water-in-silicone emulsion and will typically further comprise an emulsifier. The vehicle also may also comprise a single phase, which may be aqueous and/or organic (e.g., hydrocarbons, such as isododecane, petrolatum, and mineral oil; vegetable oils, fatty acid esters, and fatty alcohols; silicone oils, such as dimethicone, cyclomethicone and other siloxanes; and ethanol and ethanolic solutions). The extracts of Melicope elleryana will be present in the formulation in an effective amount to reduce one or more signs of skin aging, typically from about 0.0001% to about 10% (more typically, from 0.001% to about 5%) by weight of the composition. The formulations may optionally include, without limitation, a retinoid (e.g., retinoic acid, retinol, retinal, retinyl acetate, retinyl palmitate, etc.), a depigmenting agent (e.g., thiodipropionic acid), an alpha-hydroxy acid (e.g., glycolic acid), beta-hydroxy acid (e.g., salicylic acid), and/or other skin active agents (e.g., collagen stimulators, collagenase inhibitors, elastase inhibitors, N-acetyl tyrosinamide, perilla oil, cis-6-nonenol, caffeine, etc.).

In another aspect of the invention, a method is provided for improving the aesthetic appearance of human skin comprising topically applying to an area of the skin in need thereof a topical formulation comprising an effective amount of an extract of Melicope elleryana (e.g., one containing zierone and/or evodone as the most abundant constituents) having a reduced amounts of one or more volatile compounds (e.g., selected from 2-methyl-3-buten-2-ol, 3-methyl butanal, and/or 2-methyl-2-butenal), for a time sufficient to improve the aesthetic appearance of said human skin. The aesthetic improvement of human skin may be an improvement of any attribute or characteristic of skin, including without limitation:

(a) treatment, reduction, and/or prevention of fine lines or wrinkles;

(b) reduction of skin pore size;

(c) improvement in skin thickness, plumpness, and/or tautness;

(d) improvement in skin smoothness, suppleness and/or softness;

(e) improvement in skin tone, radiance, and/or clarity;

(f) improvement in procollagen, and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization;

(o) increase in skin elasticity and/or resiliency;

(p) treatment, reduction, and/or prevention of skin sagging;

(q) improvement in skin firmness; and

(r) reduction of pigment spots and/or mottled skin; and

(s) improvement of optical properties of skin by light diffraction or reflection.

In a related implementation, a method is provided for the treatment of wrinkles and/or fine lines on the skin human skin and/or reducing the severity of, reducing the number of, or preventing or forestalling the onset of, wrinkles or fine lines on human skin (typically, skin of the face) comprising topically applying to an area of the skin in need thereof (e.g., applying to a wrinkle or fine line) a topical formulation comprising an extract of Melicope elleryana (e.g., one containing zierone and/or evodone as the most abundant constituents) having a reduced amounts of one or more volatile compounds (e.g., selected from 2-methyl-3-buten-2-ol, 3-methyl butanal, and/or 2-methyl-2-butenal), for a time sufficient to improve the aesthetic appearance of said human skin. The treatment may be a least once or twice daily and may last for a period of at least four weeks, typically at least eight weeks or longer.

Further aspects, features and advantages of the present invention will be better appreciated upon a reading of the following detailed description of the invention and claims.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Therefore, specific structural and functional details disclosed herein are not to be interpreted as limiting, but merely as a representative basis for teaching one skilled in the art to variously employ the present invention.

All terms used herein are intended to have their ordinary meaning unless otherwise provided. By “cosmetically acceptable,” it is meant that a particular component is generally regarded as safe and non-toxic for application to skin at the levels employed. The term “prevent,” as used herein, includes delaying the onset of or progression of a particular sign of skin aging. The term “thin skin” includes skin that becomes thinner with chronological aging as well as prematurely thinned skin, which may be caused, for example, by photo-aging. In one embodiment, the prematurely thinned skin has been diagnosed as such by a clinician. The phrase “individual in need thereof” refers to a human that could benefit from improved dermal appearance or health, including males or females. In some embodiments, the individual in need thereof is a female.

The compositions are intended for application to the human integumentary system (including, without limitation, skin, nails, cuticles, lips, hair, etc.). In some embodiments, the integument comprises a keratinous surface. The term “skin” includes, without limitation, the lips, skin of the face, hands, arms, legs, thighs, buttocks, neck, scalp, and chest. The term “hair,” unless otherwise indicated, includes hair of the scalp, hair of the body, hair of the face, lashes, and eyebrows.

As used herein, the term “consisting essentially of” is intended to limit the invention to the specified materials or steps and those that do not materially affect the basic and novel characteristics of the claimed invention, as understood from a reading of this specification.

As used herein, 2-methyl-3-buten-2-ol is the chemical compound having CAS#115-18-4; 3-methyl butanal is the chemical compound having CAS#590-86-3, and 2-methyl-2-butenal is the chemical compound having CAS#1115-11-3.

As used herein, the term “extraction” includes, without limitation, extraction by a solvent, trituration, stripping, as well as steam and/or hydrodistillation. The term “extract,” also includes synthetic extracts. The term “extract” is not intended to limit the composition to a particular mode of extraction or isolation from the Melicope elleryana plant.

As used herein, the term “volatile” refers to a compound that readily evaporates at room temperature and atmospheric (standard) pressure. In some embodiments, the term “volatile” includes compounds having a boiling point (at atmospheric pressure) of ales than about 120° C., or less than about 110° C., or less than about 100° C., or less than about 90° C., or less than about 80° C., or less than about 70° C. In some embodiments, the term “volatile” includes at least one compound extracted from the Melicope elleryana plant. In some embodiments, the term “volatile” includes at least one compound derived from a compound extracted from the Melicope elleryana plant, by which is meant that it may be formed by reaction or decomposition during extraction process.

Without wishing to be bound by any particular theory, it is believed that the reduced off odor from the Melicope elleryana extracts of the invention is a result of the diminished content of one or more volatiles in the extract. The off odor may result from a compound extracted from the Melicope elleryana plant or the off odor may arise from decomposition or chemical reaction as a compound extracted from Melicope elleryana is subjected to solvent (e.g., water) and/or heat. The presence, absence, or relative degree of off odor can be measured by expert testers in the fragrance arts (e.g., on a 0-10 scale), or by panel testing of consumers. In some embodiments, the off odor can be quantified by dilution-to-detection techniques.

The term “low odor,” as used herein refers to an odor profile that is less intense and/or less detectable than the odor profile of a Melicope elleryana plant extract prepared by steam and hydro distillation in which the all of the distillate is collected, including the “front end,” by which is meant the lowest boiling/most highly volatile organic constituents extracted from or derived from the Melicope elleryana plant material.

The Melicope elleryana plant extracts of the invention may comprise one or more of limonene, α-gurjunene, β-elemene, β-caryophyllene, aromadendrene, α-humulene, viridiflorene, β-selinene, α-selinene δ-cadinene, zierone, globulol, viridiflorol, spathulenol, allo-evidone, evidone, and a compound with a molecular weight of 218. In other embodiments, the Melicope elleryana plant extracts of the invention may be characterized by the absence or substantial absence of any one or more of limonene, α-gurjunene, β-elemene, β-caryophyllene, aromadendrene, α-humulene, viridiflorene, β-selinene, α-selinene δ-cadinene, zierone, globulol, viridiflorol, spathulenol, allo-evidone, evidone, or a compound with a molecular weight of 218. By “substantial absence” is mean that such compound individually comprises no more than 0.01% by weight of the entire extract (without solvent), or even less than 0.001% by weight of the entire extract (without solvent).

In one embodiment, the Melicope elleryana plant extracts of the invention comprise more than 25%, more than 30%, more than 35%, more than 40%, or more than 50%, more than 60%, or more than 70% by weight evidone. In one embodiment, the Melicope elleryana plant extracts of the invention comprise less than 15%, less than 10%, less than 5%, less than 2.5%, or less than 1% by weight evidone.

In one embodiment, the Melicope elleryana plant extracts of the invention comprise more than 45%, more than 50%, more than 55%, more than 60%, or more than 65%, more than 70%, more than 75%, or more than 80% by weight zierone. In one embodiment, the Melicope elleryana plant extracts of the invention comprise less than 35%, less than 30%, less than 25%, less than 20%, less than 15%, less than 10%, less than 5%, less than 2.5%, or less than 1% by weight zierone.

In one embodiment, the Melicope elleryana plant extracts are characterized by the ability to inhibit calcineurin. In another embodiment, the Melicope elleryana plant extracts have no detectable ability to inhibit calcineurin. In one embodiment, the Melicope elleryana plant extracts are characterized by the ability to inhibit lipid accumulation in adipocytes. In another embodiment, the Melicope elleryana plant extracts lack the ability to inhibit lipid accumulation in adipocytes.

The Melicope elleryana plant from which the extract is obtained may be in any form including, but not limited to, the whole plant, a dried plant or part thereof, a ground plant or parts thereof, including but not limited to, seeds, needles, leaves, flowers, branches, branchlets, roots, bark, cones, stems, rhizomes, callus cells, protoplasts, organs and organ systems, and meristems, and/or other parts or components of the plant, or any combinations thereof. In one embodiment, the extract is derived from a Melicope elleryana plant material comprising leaves. In one embodiment, the extract is derived from a Melicope elleryana plant material comprising branchlets. In one embodiment, the extract is derived from a Melicope elleryana plant material comprising branchlets and leaves. In one embodiment, the extract is derived from the leaves and terminal branchlets of the Melicope elleryana plant.

In certain embodiments, the plant materials of the Melicope elleryana plants are ground to small particle sizes. In other embodiments, the plant materials of the Melicope elleryana plants are used in the extraction process as is, e.g., without being ground to small particle sizes. In addition, the raw plant materials may be dried to reduce water content prior to extraction. The raw plant materials may be air-dried, oven-dried, rotary evaporated under vacuum, lyophilized, or dried by any other suitable method known in the art. In other embodiments, the raw plant materials are subject to extraction without first being dried to reduce water content.

A Melicope elleryana extract may be obtained by extracting, washing, triturating, mixing, stripping, distilling, or otherwise contacting the raw plant materials with a liquid or gas.

In one embodiment, the Melicope elleryana extract is obtained by distillation. For example, the Melicope elleryana extract may be obtained by steam and/or hydro distillation. In one embodiment, the Melicope elleryana extract is obtained by a combination of steam and hydro distillation, whereby some amount of liquid water is added to a vessel containing Melicope elleryana plant material and heated. Steam is generated either from the heated water in the vessel or from injection of steam into the vessel, or from a combination of both. The vapor pressure of water vapor increases the overall vapor pressure above the plant material and or plant material/water mixture and thereby reduces the boiling point of the various constituents extracted from the plant material so that they may be vaporized at temperatures below their boiling points at standard pressure. This reduces the potential for decomposition of the extracted compounds. The desired consituents extracted from the plant material thus become entrained in the vapor phase and can be subsequently condensed back to a liquid using a condenser (e.g., a water cooled jacketed condenser, a condenser cooled by ice, dry ice, liquid nitrogen, etc.). The condensed Melicope elleryana extract may be collected in a reparatory funnel or the like where any water collected with the distillate can be removed, for example by phase separation and subsequent drying (e.g., over MgSO₄) if desired.

The extracts may also be obtained by stripping using a stripping agent. The stripping agent may be a liquid (e.g., solvent) in which some or all components of the desired extract from Melicope elleryana are either soluble, partially soluble, sparingly soluble, or insoluble. Suitable stripping agents include, but are not limited to the following: water; alcohols (such as methanol, ethanol, isopropanol, propanol, butanol and the like); glycols and glymes; ethers (such as diethyl ether, dipropyl ether, THF, and the like); esters (such as butyl acetate, ethyl acetate, and the like); ketones (such as acetone, ethyl methyl ketone, and the like); aliphatic hydrocarbons (e.g., hexane, hexanes, cyclohexane, petroleum spirits, petroleum ether, etc.), dimethyl sulfoxide; DMF, acetonitrile; methylene chloride, chloroform, and carbon tetrachloride, other organic solvents; and combinations thereof. In one embodiment, the desired extract (e.g., essential oil) from Melicope elleryana is soluble in the stripping agent. In another embodiment, the desired extract (e.g., essential oil) from Melicope elleryana is insoluble in the stripping agent. In one embodiment, the stripping agent comprises water. In one embodiment, the stripping agent is water.

In other embodiments, the Melicope elleryana extract is obtained by solvent extraction (e.g., with an organic solvent). Typically, the organic solvent extraction method involves washing the raw plant material (which may be whole or ground into small particle sizes, and optionally dried) using an organic solvent. The solvent comprise a polar aprotic solvent, a polar protic solvent, or a nonpolar solvent, or mixtures thereof. The term “polar,” as used herein, refers to a solvent having a dielectric constant of 5 or more, whereas a non-polar solvent has a dielectric constant of less than 5. In some embodiments, the dielectric constant of the solvent is greater than about 10 (e.g., from about 10-50). In some embodiments, the dielectric constant of the solvent is greater than about 15 (e.g., from about 15-50). In some embodiments, the dielectric constant of the solvent is greater than about 20 (e.g., from about 20-50). In some embodiments, the dielectric constant of the solvent is greater than about 25 (e.g., from about 25-50). In some embodiments, the dielectric constant of the solvent is from about 2-3, or from about 3-4, or from about 4-5, or from about 5-7, or from about 7-10, or from about 10-15, or from about 15-20, or from about 20-25, or from about 25-30, or from about 30-35, or from about 35-40, or from about 40-50, (where the term “about” is intended to modify bother the lower and upper end of the range).

In one embodiment, the solvent comprises, consists essentially of, or consists of a polar aprotic solvent. In one embodiment, the polar aprotic solvent comprises acetone. In one embodiment, the polar aprotic solvent comprises acetonitrile. In one embodiment, the polar aprotic solvent comprises ethyl acetate. In one embodiment, the polar aprotic solvent comprises methylene chloride. In one embodiment, the polar aprotic solvent comprises tetrahydrofuran.

In one embodiment, the solvent comprises, consists essentially of, or consists of water. For example, water may be used in combination with one or more additional liquids, which may be miscible or immiscible with water. In one embodiment, the solvent comprises water. In one embodiment, the solvent “consist essentially of” water by which is meant that additional solvents in amounts which materially alter the makeup and/or biological activity and/or organoleptic properties of the extract are excluded. The solvent system may, for example, comprise from about 1% to about 99% by weight (or from about 10% to about 90% by weight) water and from about 1% to about 99% by weight (or from about 10% to about 90% by weight) of one or more additional solvents.

In one embodiment, the solvent comprises, consists essentially of, or consists of a polar protic solvent (other than water). In one embodiment, the polar protic solvent comprises a C₁₋₈, or a C₁₋₆, alcohol, which may be, for example, primary, secondary, tertiary. Suitable alcohols include, without limitation, methanol, ethanol, propanol, isopropanol, n-butyl alcohol, isobutyl alcohol, tert-butyl alcohol, amyl alcohol, isoamyl alcohol, etc. In one embodiment, the polar protic solvent comprises methanol. In one embodiment, the polar protic solvent comprises ethanol. In one embodiment, the polar protic solvent comprises isopropanol. In one embodiment, the polar protic solvent comprises a mixture of ethanol and water. The mixture may, for example comprise from about 10% to about 90% by weight (or from about 20% to about 80% by weight) ethanol and from about 10% to about 90% by weight (or from about 20% to about 80% by weight) water. In one embodiment, the polar protic solvent comprises a C₂₋₈, or a C₂₋₆, glycol, including, without limitation, glycerin, ethylene glycol, propylene glycol, butylene glycol, etc. In one embodiment, the polar protic solvent comprises acetic acid.

In one embodiment, the solvent comprises, consists essentially of, or consists of a nonpolar solvent. In one embodiment, the nonpolar solvent comprises a C₁₋₁₂or a C₂₋₁₀ or a C₅₋₈ hydrocarbon, which may be aliphatic, aromatic, partially unsaturated, or a combination thereof. In one embodiment, the nonpolar solvent comprises an aliphatic hydrocarbon, including without limitation pentane, hexane (including cyclohexane), heptane, and octane. In one embodiment, the nonpolar solvent comprises hexane. In one embodiment, the nonpolar solvent comprises benzene. In one embodiment, the nonpolar solvent comprises toluene. In one embodiment, the nonpolar solvent comprises xylene. In some embodiment, the nonpolar solvent may comprise a halogenated hydrocarbon, such as chloroform or carbon tetrachloride. In one embodiment, the nonpolar solvent comprises a di-C₁₋₆-alkyl ether, such as diethyl ether, di-isopropyl ether, dibutyl ether, etc., including mixed ethers.

In one embodiment, the solvent comprises, consists essentially of, or consists of liquid CO₂ or supercritical CO₂.

An extracting machine may be used for solvent extraction as is well known in the field. Typically, raw plant materials are pushed slowly into the extracting machine by a thruster. Solvent, such as an organic solvent (e.g., ethanol), may be added into the machine through a solvent inlet at the top of a waste discharge outlet. Due to the difference in gravity and equilibrium, the solvent flows toward the raw plant material inlet, soaks the materials and flows out from the opposite side of the solvent inlet. Since the plant materials and the solvent move in opposite directions against each other (e.g., countercurrent), the plant materials are constantly immersed in a solution that contains a low-concentration of extract. As a result of equilibrium, high yield of plant constituent(s) may be achieved by continuously extracting the plant material against the low-concentration solution.

An extraction time suitable to extract the Melicope elleryana plant constituents is used, typically between about 1 minute and 24 hours, or between about 1-10 hours, in one embodiment between about 2-8 hours, and in another embodiment between about 3-6 hours. The temperature of extraction is typically between about 30° C.-100° C., in one embodiment between about 40° C.-70° C., and in another embodiment between about 50° C.-60° C. The collected extract may then be fine-filtered to remove debris, and may be used directly, or may be concentrated, for example by distilling or evaporating the solvent or by other conventional processing. For example, the extract may be rotary evaporated under vacuum or lyophilized.

Similarly, aqueous-organic solvent extraction may involve, for example, initially collecting raw materials from the Melicope elleryana plant, which may be whole or ground into small particle sizes and soaking the ground plant material in aqueous solution that is acidic or alkaline, depending on the solubility and stability of the desired extract under acidic or alkaline (basic) conditions. For extraction under acidic conditions, an acid, such as hydrochloric acid or sulfuric acid is added to water, e.g., at a concentration of about 3% (w/v). For extraction under alkaline conditions, an alkali such as sodium hydroxide or sodium carbonate is added to water. The extraction time and temperature of extraction are typically similar to that used in the organic solvent extraction method described above.

The extracts of the invention may be fine-filtered to remove debris (e.g., plant debris). Alkaline agents (e.g., ammonia) or acidifying agents (e.g., sulfuric acid) may be added to the extract to neutralize the solution by adjusting the pH, depending on the acidity or alkalinity of the collected extract. The aqueous extract may be used directly, in concentrated or dried form. Alternatively, organic solvent (including, without limitation, any of the extraction solvents discussed above) may then be added to the neutralized solution to transfer the extract from an aqueous phase to an organic phase and subsequently isolated by solvent removal.

It should also be noted that different plants containing different constituents can be mixed and extracted together with Melicope elleryana. This process of mixed extraction can be used for extracting those plants containing constituents with similar solubility as Melicope elleryana in the solvent used for extraction, such as ethanol and/or water.

In another embodiment, the Melicope elleryana extract as used herein, also includes “synthetic” extracts, i.e., various combinations of Melicope elleryana plant components and/or constituents that are combined to substantially mimic the composition and/or activity of a Melicope elleryana plant extract of natural origin. The synthetic extracts may have substantially the same number of active components as a natural Melicope elleryana plant material. The correspondence of the numerical incidence of actives between the synthetic extracts and the natural Melicope elleryana plant material may also be described in terms of “percent commonality.” The synthetic extract may have about 50 percent or more commonality to the chemical composition of a plant or natural extract. In other words, the synthetic extract may have about 50 percent or more of the active ingredients found in the plant or a natural extract. More typically, the chemical composition of the synthetic extract may have about 70 percent or more commonality to the chemical composition of a plant or a natural extract. Optimally, a synthetic extract may have about 90 percent or more commonality to the chemical composition of a plant or a natural extract. Alternatively, the synthetic extract may have at least 50% (or at least about 70% or at least about 90%) commonality to the natural extract on a mass basis, by which is meant that at least 50% (or at least about 70% or at least about 90%) of the mass of the synthetic extract is identical to that of the natural extract.

The Melicope elleryana plant extracts of the invention may be formulated in cosmetically acceptable vehicles. The cosmetic formulation of the invention may comprises one or more of a film forming polymer, a thickener, a pH adjuster, a preservative, an emulsifier, a gelling agent, an antioxidant, a fragrance, a colorant, and the like. The vehicle may comprise a water-in-oil, oil-in-water, silicone-in-water, or water-in-silicone emulsion and will typically further comprise an emulsifier. The effective amount of the compound will typically be from about 0.00001% to about 10% (more typically, from 0.001% to about 5%) by weight of the composition. The formulations may optionally include a retinoid, for example retinoic acid, retinol, retinal, retinyl acetate, fatty acid ester of retinol, such as retinyl palmitate, to name a few. The composition may optionally further comprise an alpha-hydroxy acid (e.g., glycolic acid) and/or a beta-hydroxy acid (e.g., salicylic acid) in amounts effective to improve the appearance of skin.

In a related aspect, methods are provided for enhancing the appearance of human skin comprising topically applying to an area of the skin in need thereof (e.g., sagging skin, thinning skin, skin suffering from wrinkles and fine lines, etc.) a topical composition comprising an extract of the Melicope elleryana plant according to any embodiment of the invention, in a cosmetically acceptable vehicle, for a time sufficient to improve the appearance thereof. The treatment may be a least once or twice daily and may last for a period of at least four weeks, typically at least eight weeks or longer.

The compositions can include a cosmetically acceptable vehicle. Such vehicles may take the form of any known in the art suitable for application to skin and may include, but are not limited to, water; vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, hydrocarbon oils such as mineral oil, petrolatum, isoeicosane and polyisobutene; polyols such as propylene glycol, glycerin, butylene glycol, pentylene glycol and hexylene glycol; liposomes; waxes; or any combinations or mixtures of the foregoing.

The vehicle may comprise an aqueous phase, an oil phase, an alcohol, a silicone phase or mixtures thereof and may be in the form of an emulsion. Non-limiting examples of suitable emulsions include water-in-oil emulsions, oil-in-water emulsions, silicone-in-water emulsions, water-in-silicone emulsions, glycerin-in-oil emulsions, wax-in-water emulsions, water-oil-water triple emulsions or the like. The emulsion may include an emulsifier, such as a nonionic, anionic or amphoteric surfactant, or a gelling agent.

In one embodiment, the topical composition will have a pH range from 1 to 11, with a pH in the range of from 2 to 8 being typical. Suitable pH adjusters such as citric acid and triethanolamine may be added to bring the pH within the desired range.

In one embodiment of the invention, the compositions may include additional skin actives, including but not limited to, retinoids, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors.

The composition may comprise additional active ingredients having anti-aging benefits, as it is contemplated that synergistic improvements may be obtained with such combinations. Exemplary anti-aging components include, without limitation, botanicals (e.g., Butea frondosa extract); phytol; thiodipropionic acid (TDPA) and esters thereof; retinoids (e.g., 9-cis retinoic acid, 13-cis retinoic acid, all-trans retinoic acid and derivatives thereof, phytanic acid, retinol (Vitamin A) and esters thereof, such as retinol palmitate, retinol acetate and retinol propionate, and salts thereof and others); hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few.

Exemplary retinoids include, without limitation, retinoic acid (e.g., all-trans or 13-cis), and derivatives thereof, retinaldehyde, retinol (Vitamin A) and esters thereof, such as retinol palmitate, retinol acetate and retinol propionate, and salts thereof. Particular mention may be made of retinol. It is contemplated that combinations of the compounds of Formula I(a) or I(b) with any of these retinoids will provide enhanced or synergistic improvements to skin. The retinoids will typically be included in amounts from about 0.0001% to about 5% by weight, more typically from about 0.01% to about 2.5% by weight, or from about 0.1% to about 1.0% by weight. Compositions according to this embodiment will typically include an antioxidant such as ascorbic acid and/or BHT and/or a chelating agent such as EDTA or a salt thereof

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; a sunscreen, such as avobenzone; an exfoliating agent; and an antioxidant.

Suitable exfoliating agents include, for example, alpha-hydroxy acids, beta-hydroxy acids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.01% to about 20% by weight of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. In one particular embodiment, the inventive compositions will include a combination of retinol, TDPA or an ester thereof (notably, dilauryl thiodipropionic acid), and an alpha hydroxyl acid (glycolic acid) and/or beta hydroxyl acid (salicylic acid). Compositions of the present invention may comprise an antioxidant, which may comprise from about 0.001 wt % to about 10 wt %, or from about 0.01 wt % to about 5 wt %, of the total weight of the composition.

Other conventional additives include: vitamins, such as tocopherol and ascorbic acid; vitamin derivatives such as ascorbyl monopalmitate; thickeners such as hydroxyalkyl cellulose; gelling agents; structuring agents; metal chelating agents such as EDTA or salts thereof; pigments; colorants; and pH adjusters. The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders.

The composition may be formulated in a variety of product forms, such as, for example, an emulsion, lotion, cream, serum, spray, aerosol, cake, ointment, essence, gel, paste, patch, pencil, towelette, mask, stick, foam, elixir, concentrate, and the like, particularly for topical administration. The composition is typically formulated as an emulsion, lotion, cream, ointment, serum or gel.

Generally, the improvement in the condition and/or aesthetic appearance is selected from the group consisting of: reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; preventing, reducing, and/or treating hyperpigmentation or hypopigmentation; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; slowing or halting skin thinning; increasing skin elasticity and/or resiliency; enhancing exfoliation; improving microcirculation; decreasing and/or preventing cellulite formation; and any combinations thereof. In some embodiments, each of the forgoing is associated with female skin.

In some embodiments, the cosmetic compositions of the invention will be applied to the skin in an amount from about 0.001 to about 100 mg/cm², more typically from about 0.01 to about 20 mg/cm², or from about 0.1 to about 10 mg/cm².

It is also contemplated that the compositions of the invention will be useful for treating thin skin by topically applying the composition to thin skin of an individual in need thereof. “Thin skin” is intended to include skin that is thinned due to chronological aging, menopause, or photo-damage and skin that is thinning prematurely. In some embodiments, the treatment is for thin skin in men, whereas other embodiments treat thin skin in women, pre-menopausal or post-menopausal, as it is believed that skin thins differently with age in men and women, and in particular in women at different stages of life.

The method of the invention may be employed prophylactically to forestall aging including in individuals that have not manifested signs of skin aging, most commonly in individuals under 25 years of age. The method may also reverse or treat signs of aging once manifested as is common in individuals over 25 years of age, or to slow the progression of dermatological aging in such individuals.

In one embodiment, the compositions of the invention are applied to human skin to reduce sebum production or improve the appearance of skin affected by cellulite, and/or reduce unwanted lipogenesis or increase lipolysis. In this embodiment, the extracts can be formulated in cosmetically acceptable vehicles (as described herein) and may include one or more additional agents such as anti-acne ingredients (e.g., salicylic acid, benzoyl peroxide and other peroxides, sulfur, retinoids, etc.) in the case of a facial composition, or, in the case of a cellulite treatment, the formulation may comprise any ingredients suitable for treatment of cellulite, including without limitation, perilla oil and other unsaturated fatty oils and omega-3 fatty acids such as alpha-linolenic acid; caffeine; theophylline; xanthines; retinoids (e.g., retinol); and the like. A cellulite treatment according to the invention will typically be applied topically to skin suffering from cellulite, including skin of the buttocks and thighs for a period of time sufficient to improve the appearance thereof, including for example, daily treatment for at least four weeks, at least eight weeks, at least twelve weeks, or longer.

In another embodiment, the compositions of the invention are applied to human skin for depigmentation, including reducing areas of unwanted pigmentation, such as hyperpigmentation, including age spots and freckles. In this embodiment, the cosmetic formulations may include one or more additional agents that combat pigmentation or hyperpigmentation, including tyrosinase inhibitors and/or melanosome transfer inhibitors. Special mention may be made of thiodipropionic acid and esters thereof (notably, di-lauryl esters); hydroquinone and the monobenzyl ether thereof; hydroquinone-beta-D-glucopyranoside; retinoids (e.g., retinoic acid); tretinoin; azelaic acid; Kojic acid (5-hydroxy-4-pyran-4-one-2-methyl); Mequinol (4-hydroxyanisole); Niacinamide; soy protein and other serine protease inhibitors; paper mulberry extract; Glabridin (licorice extract); Arctostaphylos patula and Arctostaphylos viscida extracts; Magnesium-L-ascorbyl-2-phosphate (MAP); 4-Isopropylcatechol; Aleosin; N-acetyl-4-S-cysteaminylphenol and N-propionyl-4-S-cysteaminylphenol; N-acetyl glucosamine; and Tranexamic acid (trans-4-aminomethylcyclohexanecarboxylic acid); to name a few.

In another embodiment, the extracts are intended for oral use, including for pharmaceutical use. Pharmaceutical formulations will include pharmaceutically acceptable carriers (i.e., diluents and excipients). The pharmaceutical compositions may be included in solid dosage forms, including compressed tablets, capsules, or caplets, or in liquid or powder forms. Pharmaceutical and/or other oral dosage forms will typically include from about 1 mg to about 2,000 mg, or from about 100 mg to about 1,000 mg of the extract.

### EXAMPLES

The following example describes specific aspects of the invention to illustrate the invention but should not be construed as limiting the invention, as the example merely provide specific methodology useful in the understanding and practice of the invention and its various aspects.

### Example 1

### Steam Distillation of Melicope elleryana

Melicope elleryana has previously been extracted by a combination of steam distillation and hydro-distillation, using partial immersion of the biomass in boiling and steam. The heat opens the oil glands in the plant and allows the essential oil and water to mix. Due to the increased vapor pressure in the system do to boiling water, there is a reduction in boiling point of the organic constituents and essential oil, which would normally have a boiling point of greater than 200° C., and caused to boil at less than 100° C. Leaves and terminal branchlets were cut from stands of Melicope elleryana growing wild on the north coast of New South Wales (NSW), Australia. Approximately 2.102 Kg of leaf biomass was loaded loosely into a 20 liter reaction vessel set up as a distillation unit with a jacketed, water-cooled condenser draining into a 500 ml separating flask. 4 liters of hot water were added to the vessel and additional heat added via a hot plate. The flow rate of the condenser water was adjusted to give a distillate temperature of at least 50° C. in the separating funnel. The essential oil floated on the water in the separating funnel. At approximately 1 hour intervals the water was drained off and returned to the vessel. The distillation was stopped after 8.5 hours when no further oil was distilled. The water in the separating flask was drained and the essential oil tapped off. The 2.102 Kg of leaf biomass produced 3.35 g of essential oil, a yield of 0.16%. The essential oil was found to have a relative density at 20° C. of 0.950 and a Refractive Index of 1.510 at 20° C.

### Example 2

### Redistillation of Melicope elleryana

An extract of Melicope elleryana, for example an extract obtained in example 1, is redistilled by loading either the entire distillate (i.e., including water) or the separated essential oil layer into a distillation unit equipped with a jacketed, water-cooled condenser draining into a separating flask. Hot water and/or steam are added to the vessel and the mixture is heated to a boil. The initial condensate collected in the separating funnel is richer in 2-methyl-3-buten-2-ol, 3-methyl butanal, and/or 2-methyl-2-butenal than the starting material (i.e., the extract provided in example 1). The condensate is discarded and the remaining material in is used as is or is further purified as desired, for example, by continuing the distillation. In a variant of the method, the vapors which initially form upon boiling are not condensed but are exhausted without collection. These initial vapors are believed to be richer in 2-methyl-3-buten-2-ol, 3-methyl butanal, and/or 2-methyl-2-butenal than the starting material (i.e., the extract provided in example 1). After some amount of vapor rich in the off odor compounds is exhausted, the distillation is continued and the remaining vapors, or at least some fraction thereof, are condensed and collected.

### Example 3

### GC Analysis of Headspace

The volatile constituents of an extract of Melicope elleryana may be evaluated by headspace analysis as follows. 500 mg of extract (neat) is placed into a airtight 10 ml vial and placed in an oven at 100° F. for one hour. A 0.5 ml sample of the headspace (gas phase) over the sample was withdrawn using a gas syringe and injected into the inlet port of GC/MS having the following operating parameters:

Column: HP-5 30M capillary, 30 m, 320 microns I.D. —0.1 micron film

Inlet: 200° C.

GC/MS scan at 40-200 amu total ion

Total run time: 5 minutes

Injection volume: 0.5 cc

Direct injection splitless

Initial flow: 1.4 ml/min

Pressure: 3.34 PSI

An extract prepared substantially according to example 1 was subjected to headspace testing as outlined above. The GC showed three major elution peaks with retention times between 1-3 minutes at approximately 1.35 minutes, about 1.59 minutes, and about 2.30 minutes. The integrated areas of the peaks, as a percentage of the total area under the curve, were about 58%, about 19%, and about 23%, respectively. The mass spectra of each peak was obtained and compared to the Wiley database. The peaks were thereby identified as 2-methyl-3-buten-2-ol, 3-methyl butanal, and 2-methyl-2-butenal, respectively.

### Example 4

### GC Analysis of Low Odor Extract

The constituents of an extract of Melicope elleryana may be evaluated by gas chromatography (GC) analysis as follows. 0.05 g of extract (neat) is diluted volumetrically to 50 ml in acetone. The sample (2 microliters) was injected into the inlet port of GC/MS having the following operating parameters:

Column: HP-5 30M capillary 320 microns I.D.—0.1 micron film

Inlet: 250° C.

GC/MS scan at 40-400 amu total ion

Total run time: 30 minutes

Injection volume: 2 μl

Direct injection, splitless

Initial flow: 1.4 ml/min

Pressure: 3.37 PSI

Solvent delay 2.00 minutes

Oven 100° C. hold 2 minutes, then 3° C. to 240° C.

The GC showed several elution peaks (where the retention time RT in minutes is indicated before the parenthetical and the integrated area under each peak is expresses as a percentage of the total area within the parenthetical; each value is approximate), comprising the following: 8.2 (1.1%), 8.7 (0.9%), 8.8 (3.2%), 9.0 (3.7%), 9.2 (0.9%), 9.6 (0.9%), 9.7 (0.8%), 10.6 (1.2%), 11.3 (3.6%), 12.1 (1.0%) 14.1 (31.3%), 15.0 (1.6%), 16.8 (3.2%), 18.1 (4.4%), 20.0 (0.9%), 24.6 (5.6%), and 25.4 (35.8%). The peaks at 14.1 and 25.4 minutes are believed to be zierone and evidone, respectively.

### Example 5

### GC/MS Quantification of 2-Methyl-3-Buten-2-Ol

The 2-methyl-3-buten-2-ol content of various extracts of Melicope elleryana was evaluated by GC/MS analysis as follows. Test samples of Melicope elleryana extract (0.1 g) were dissolved in 10 ml in acetone. Standard solutions of 3-buten-2-ol were prepared by dissolving it in acetone at concentrations of 1.4, 14, and 140 ppm. For each standard solution and for samples, 2 microliters were injected into the inlet port of GC/MS having the following operating parameters:

Column: HP-5 30M capillary 320 microns I.D. —0.1 micron film

Inlet: 250° C.

GC/MS scan at 43, 57 and 71 amu

Total run time: 35 minutes

Injection volume: 2 μl

Direct injection, splitless

Initial flow: 1.7 ml/min

Pressure: 2.39 PSI

Oven 40° C. hold 5 minutes, then 20° C./min to 240° C. for 20 min

The MS fragment at 71 amu was isolated for measurement. The height of the 71 amu peak was determined for the three standards and a linear calibration curve was prepared. The 71 amu MS peak for the Melicope elleryana materials was measured and compared against the standard curve. The resultant value was divided by five based on data from the Wiley MS database which shows that the 71 amu peak for 3-buten-2-ol is five-fold stronger than for 2-methyl-3-buten-2-ol. Based on this experiment, the amount of 2-methyl-3-buten-2-ol in a Melicope elleryana extract that was obtained by steam distillation and subsequently re-distilled to remove volatiles was found to be 0.003% by weight. The experiment was repeated with a sample of Melicope elleryana extract that was prepared by a single steam/hydrodistallation wherein the “front end” (e.g., a fraction of high volatility components that was the first time-wise to volatilize) was removed. That sample also showed very low content (0.007% w/w) of 2-methyl-3-buten-2-ol. A third sample of Melicope elleryana extract that was prepared by a single steam/hydrodistallation without removal of volatiles was also tested. That sample also was characterized by an objectionable odor profile and showed relatively high content (0.14% w/w) of 2-methyl-3-buten-2-ol.

In some embodiments, the Melicope elleryana extracts of the invention will have less than 0.10% w/w of 2-methyl-3-buten-2-ol. In other embodiments, the Melicope elleryana extracts of the invention will have less than 0.08%, or less than 0.07%, or less than 0.06%, or less than 0.05%, or less than 0.04%, or less than 0.03%, or less than 0.02%, or less than 0.01 w/w of 2-methyl-3-buten-2-ol. In other embodiments, the Melicope elleryana extracts of the invention will have less than 0.005% w/w of 2-methyl-3-buten-2-ol.

In some embodiments, the Melicope elleryana extracts of the invention will have less than 0.10% w/w of 3-methyl butanal. In other embodiments, the Melicope elleryana extracts of the invention will have less than 0.08%, or less than 0.07%, or less than 0.06%, or less than 0.05%, or less than 0.04%, or less than 0.03%, or less than 0.02%, or less than 0.01 w/w of 3-methyl butanal. In other embodiments, the Melicope elleryana extracts of the invention will have less than 0.005% w/w of 3-methyl butanal.

In some embodiments, the Melicope elleryana extracts of the invention will have less than 0.10% w/w of 2-methyl-2-butenal. In other embodiments, the Melicope elleryana extracts of the invention will have less than 0.08%, or less than 0.07%, or less than 0.06%, or less than 0.05%, or less than 0.04%, or less than 0.03%, or less than 0.02%, or less than 0.01 w/w of 2-methyl-2-butenal. In other embodiments, the Melicope elleryana extracts of the invention will have less than 0.005% w/w of 2-methyl-2-butenal.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A cosmetic formulation for improving the aesthetic appearance of human skin comprising a cosmetically acceptable vehicle, and an effective amount of a low odor extract of Melicope elleryana, wherein said extract has been processed to remove one or more volatile constituents.

2. The cosmetic formulation according to claim 1, wherein said low odor extract is obtained by: (1) heating the Melicope elleryana plant, or a portion thereof, in the presence of water and/or steam, to produce (i) a first fraction of vapors comprising at least one volatile constituent, and (ii) a second fraction of vapors comprising a relatively lower abundance of said one or more volatile constituents than in said first fraction of vapors, and (2) condensing said second fraction of vapors.

3. The cosmetic formulation according to claim 2, wherein said Melicope elleryana plant, or a portion thereof, includes one or more of leaves, branchlets, flowers, and seeds.

4. The cosmetic formulation according to claim 2, wherein at least one of said one or more volatile compounds has a boiling point less than 120° C.

5. The cosmetic formulation according to claim 2, wherein said first fraction of vapors comprises one or more of 2-methyl-3-buten-2-ol, 3-methyl butanal, and 2-methyl-2-butenal.

6. The cosmetic formulation according to claim 1, wherein said low odor extract is obtained by heating a composition comprising a first extract of Melicope elleryana to separate therefrom a first fraction of vapors comprising at least one volatile constituent.

7. The cosmetic formulation according to claim 6, wherein at least one of said one or more volatile compounds has a boiling point less than 120° C.

8. The cosmetic formulation according to claim 6, wherein said at least one volatile constituent comprises one or more of 2-methyl-3-buten-2-ol, 3-methyl butanal, and 2-methyl-2-butenal.

9. The cosmetic formulation according to claim 6, wherein said composition is heated to produce a second fraction of vapors comprising evodione and/or zierone in a relatively greater abundance than in said first fraction of vapors, and wherein said second fraction of vapors are thereafter condensed.

10. The cosmetic formulation according to claim 1, wherein said low odor extract is obtained by contacting a first extract of Melicope elleryana with activated carbon.

11. The cosmetic formulation according to claim 1, wherein said cosmetically acceptable vehicle comprises a water-in-oil or oil-in-water emulsion and further comprises an emulsifier.

12. The cosmetic formulation according to claim 1, wherein said effective amount comprises from about 0.0001% to about 10% by weight of said composition.

13. The cosmetic formulation according to claim 1, further comprising a retinoid.

14. The cosmetic formulation according to claim 1, wherein said composition further comprises an alpha-hydroxy or beta-hydroxy acid.

15. A cosmetic formulation for improving the aesthetic appearance of human skin comprising a cosmetically acceptable vehicle, and an effective amount of an extract of Melicope elleryana, wherein said extract has been obtained by extraction of the Melicope elleryana plant, or any portion thereof, with a solvent comprising a polar solvent.

16. The cosmetic formulation according to claim 15, wherein said polar solvent is a polar aprotic solvent.

17. The cosmetic formulation according to claim 15, wherein said polar solvent is a protic solvent other than water, alone or in combination with water.

18. The cosmetic formulation according to claim 17, wherein said mixture comprises from about 10% to about 90% by weight ethanol and from about 10% to about 90% by weight water.

19. A cosmetic formulation for improving the aesthetic appearance of human skin comprising a cosmetically acceptable vehicle, and an effective amount of an extract of Melicope elleryana, wherein said extract has been obtained by extraction of the Melicope elleryana plant, or any portion thereof, with a solvent comprising a nonpolar solvent.

20. A cosmetic formulation for improving the aesthetic appearance of human skin comprising a cosmetically acceptable vehicle, and an effective amount of an extract of Melicope elleryana, wherein said extract has been obtained by extraction of the Melicope elleryana plant, or any portion thereof, with liquid CO₂ or supercritical CO₂.
